(19) United States
(12) Patent Application Publication
(10) Pub. No.: US 2003/0036065 A1
(43) Pub. Date: Feb. 20, 2003

(54) Method and system for array signal generation and amplification

(76) Inventor: Robert Gellibolian, Fremont, CA (US)

Correspondence Address: AGILENT TECHNOLOGIES, INC., Intellectual Property Administration, Legal Department, DL429, P.O. Box 7599, Loveland, CO, 80537-0599, US

(21) Appl. No.: 09/932,728

(22) Filed: Aug. 17, 2001

(51) Int. Cl. C12Q 1/68; G01N 33/53; G01N 33/542; G06F 19/00; G01N 33/48; G01N 33/50

(52) U.S. Cl. 435/6; 435/7.9; 702/19; 702/20

(57) ABSTRACT

A method and system for signal generation and signal amplification from an array containing bound, unlabeled target molecules. Following exposure of the array to a sample solution containing unlabaled target RNA molecules, blunt ends are generated on each probe/target double-stranded hybrid labeled primer oligonucleotide linker is then bound to the blunt ends. Next, in an iterative, inner process, additional layers of labeled oligonucleotide, linkers are added, shell-by-shell, to form a dendrimer-like molecular complex bound through the oligonucleotide linker to the probe/target hybrid.

### TECHNICAL FIELD

The present invention relates to the analysis of molecular-array-based hybridization experiments, diagnostic procedures, and other analytical procedures and, in particular, to a method and system for detecting target molecules hybridized to probe molecules within the features of an array using labeled oligonucleotide linkers to create dendrimer-like, branching molecular entities covalently bound to hybridized target/probe pairs.

### BACKGROUND OF THE INVENTION

The present invention is related to processing of data scanned from arrays. Array technologies have gained prominence in biological research and are likely to become important and widely used diagnostic tools in the healthcare industry. Currently, molecular-array techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrate the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features are prepared by various manufacturing processes. The array 402 in FIG. 4, and in subsequent FIGS. 5-7, has a grid-like two-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemoluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound labeled DNA molecules. Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemoluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

Array-based hybridization techniques allow extremely complex solutions of DNA molecules to be analyzed in a single experiment. An array may contain from hundreds to tens of thousands of different oligonucleotide probes, allowing for the detection of a subset of complementary sequences from a complex pool of different target DNA or RNA polymers. In order to perform different sets of hybridization analyses, arrays containing different sets of bound oligonucleotides are manufactured by any of a number of complex manufacturing techniques. These techniques generally involve synthesizing the oligonucleotides within corresponding features of the array through a series of complex iterative synthetic steps.

As pointed out above, array-based assays can involve other types of biopolymers, synthetic polymers, and other types of chemical entities. For example, one might attach protein antibodies to features of the array that would bind to soluble labeled antigens in a sample solution. Many other types of chemical assays may be facilitated by array technologies. For example, polysaccharides, glycoproteins, synthetic copolymers, including block coploymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Once the labeled target molecule has been hybridized to the probe on the surface, the array may be scanned by an appropriate technique, such as by optical scanning in cases where the labeling molecule is a fluorophore or by radiometric scanning in cases where the signal is generated through a radioactive decay of labeled target. In the case of optical scanning, each different wavelength at which an array is scanned produces a different signal. Thus, in optical scanning, it is common to describe the signal produced by scanning in terms of the color of the wavelength of light employed for the scan. For example, a red signal is produced by scanning the array with light having a wavelength corresponding to that of visible red light.

Scanning of a feature by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use.

Although the above-described array-based experimental and diagnostic procedures have been successfully applied to determine gene expression levels in complex organisms, to sequence DNA and RNA, detect the presence of target molecules in complex solutions, and to perform other such analytical tasks, the commonly employed techniques suffer from certain drawbacks. First, target molecules must be labeled with chromophores, radionuclides, or other types of signal-emitting chemical entities. Labeling of target molecules may add significant cost and time overheads to experimental procedures, and may introduce significant sources of experimental error. One source of error is that labeling often involves incorporating fluorophore-attached nucleotides during amplification of messenger RNA. The bulky nature of these fluorophores may result in a significant reduction in the hybridization efficiency of the labeled target molecules to the probes on the surface of the array. Furthermore, the amount of signal-emitting entities, or label, bound to probe molecules within an array feature is directly proportional to the amount of target molecules hybridized to probe molecules. When target/probe hybridization is weak or inhibited by the presence of competing molecules that may hybridize to probe molecules, when the signal produced by the label is altered, attenuated, or masked, or when the concentration of the target molecules is relatively low, the signal-to-noise ratio for the array feature may decrease below an acceptable statistical threshold, despite hybridization of target molecules to features within the array. Various techniques have been tried to amplify signals emitted from labeled target molecules, but currently employed amplification techniques also suffer from various deficiencies, including cross reactivity of amplifying molecules with unrelated probe and target molecules, steric hindrance in binding amplifying entities to probe/target pairs, and additional expense and time required to amplify signals during array analysis. For these reasons, designers, manufacturers and users of arrays have recognized the need for alternative signal generation amplification techniques.

### SUMMARY OF THE INVENTION

The present invention is related to signal generation and amplification from features of an array. Unlike commonly employed array-based experimental, diagnostic, and other analytical procedures, the present invention allows for signal generation following hybridization of unlabeled target molecules to probes bound to features of an array. Following exposure of an array to a sample solution containing unlabeled target molecules, and following hybridization of the unlabeled target molecules to probes, extraneous and unbound molecules are rinsed from the surface of the array leaving behind probe/target pairs bound together by hydrogen bonding, stacking, and electrostatic interactions. In a described embodiment, amplified RNA target molecules hybridize to oligonucleotide probe molecules.

Following hybridization of target molecules to probes, and rinsing, one of various techniques is used to generate blunt ends on each probe/target double-stranded hybrid. Next, a primer oligonucleotide linker is ligated to the blunt ends. The primer oligonucleotide linker comprises two partially complementary polynucleotides that partially hybridize to form a double-stranded region and two single-stranded, noncomplementary arms. Next, in an iterative inner process, additional layers of labeled oligonucleotide linkers are added, shell-by-shell, to a dendrimer-like molecular complex bound through the primer oligonucleotide linker to the probe/target hybrid. In the iterative process, a first set of labeled oligonucleotide linkers with three single-stranded arms are hybridized via the third single-stranded arm to free, unhybridized, single-stranded arms of the previously added oligonucleotide linkers, and then covalently bound by a DNA-ligase-mediated reaction. Each newly added labeled oligonucleotide linker provides two new, unhybridized, single-stranded arms. Then, a second set of oligonucleotide linkers having free single-stranded arms is hybridized through their third arms to the unhybridized, single-stranded arms of the first set of oligonucleotide linkers and then covalently bound. Additional layers of oligonucleotide linkers can be hybridized to the growing, dendrimer-like molecular complex bound to the probe/target pair by alternating, successive applications of the first set of labeled oligonucleotide linkers and the second set of labeled oligonucleotide linkers. Each successive application of labeled oligonucleotide linkers may double the amount of label bound to a probe/target pair, amplifying the signal exponentially.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIG. 8 shows a small portion of an array prior to exposure of the array to a sample solution containing target molecules.

FIG. 9 illustrates exposure of the portion of the array to a sample solution containing target molecules.

FIG. 10 illustrates the portion of the array following rinsing of the sample solution containing target molecules from the surface of the array.

FIG. 11 illustrates one possible first step of a signal generating technique provided by the present invention.

FIG. 12 illustrates a second alternative first step of a signal generating technique provided by the present invention.

FIG. 13 shows the blunt-ended target/probe double-stranded duplex following probe elongation or target 5′ single-stranded-arm removal.

FIG. 14 shows the signal-emitting chemical entities used to generate signals and amplify signals from array features containing target/probe hybrids.

FIG. 15 illustrates attachment of a primary oligonucleotide linker to the blunt-ended, double-stranded target/probe hybrid bound to a feature of an array.

FIG. 16 shows a first amplification step in the described embodiment of the present invention.

FIG. 17 shows the resulting topographically Y-shaped molecular entity following ligation.

FIG. 18 illustrates addition of another layer of oligonucleotide linkers to the branching molecular complex covalently linked to the target/probe hybrid.

FIG. 19 shows the complex branching molecular complex shown in FIG. 18 following ligation.

FIG. 20 abstractly represents additional layers, or shells, of oligonucleotide linkers added to further increase the size of the complex, branching molecular complex covalently bound to the target/probe hybrid.

FIG. 21 is a flow-control diagram that describes various alternative embodiments of the present invention in an algorithmic fashion.

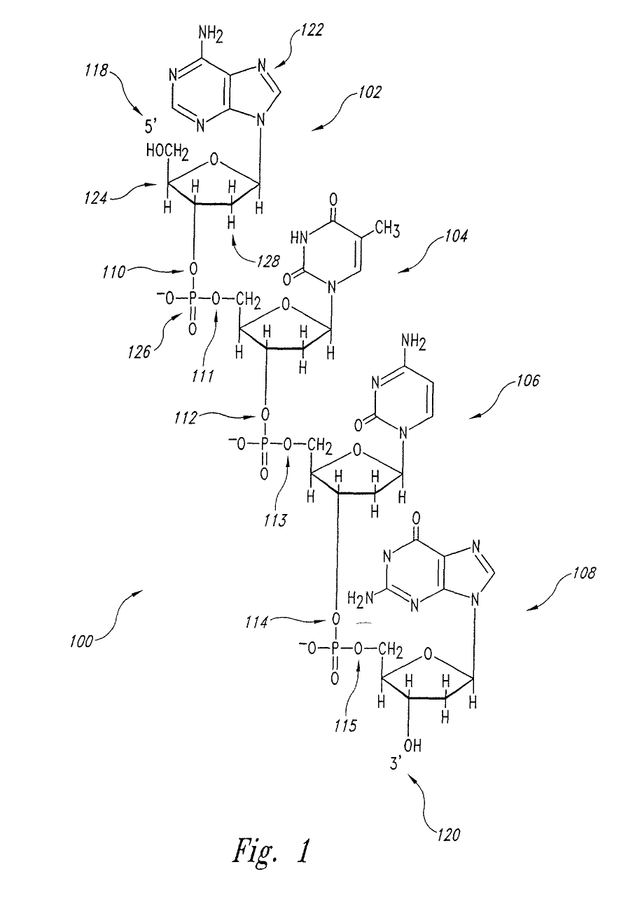

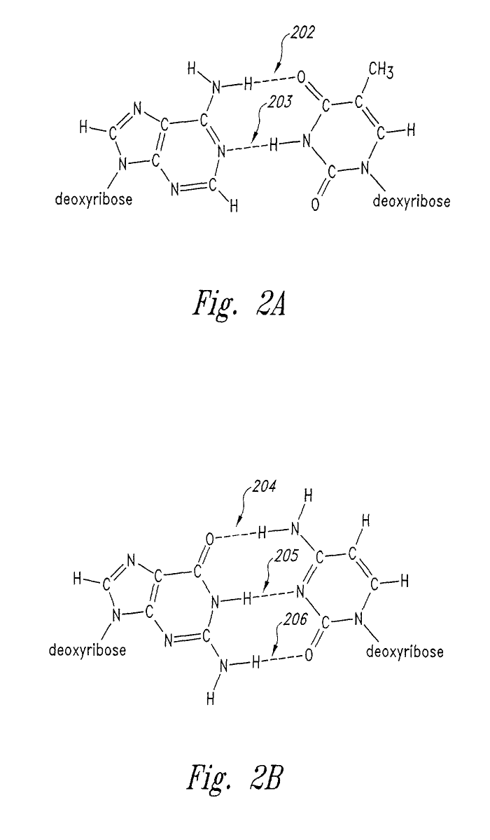

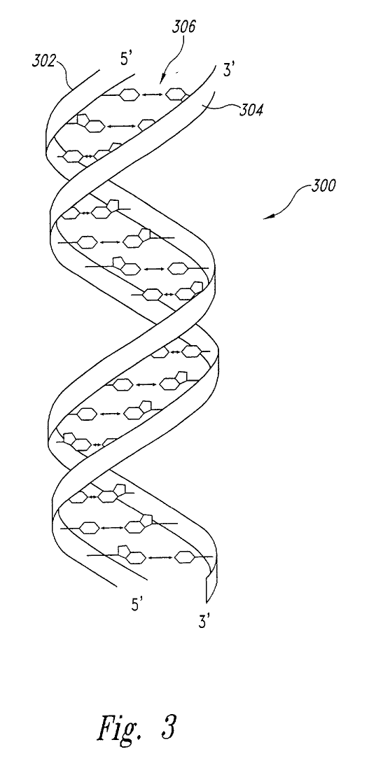

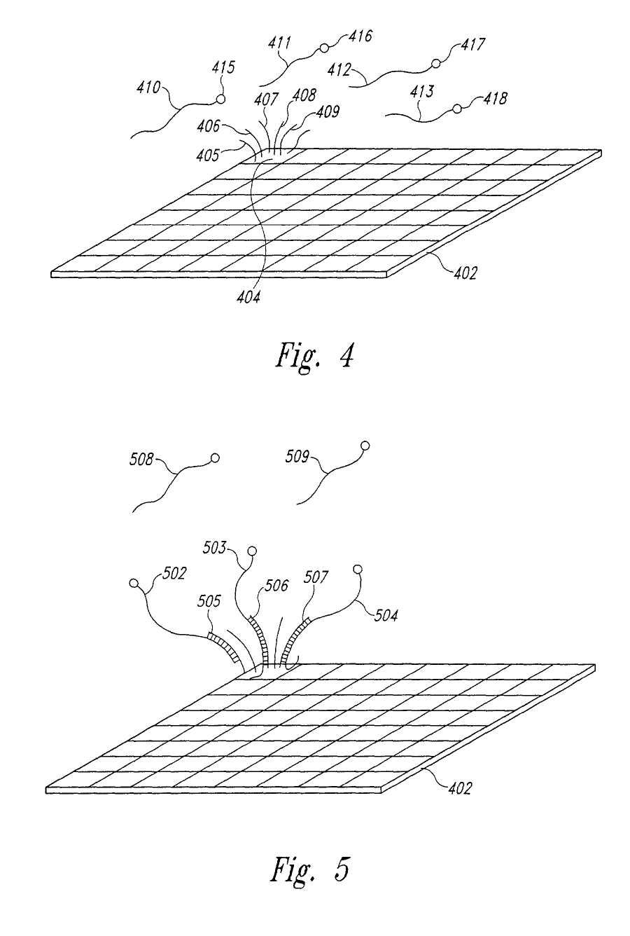

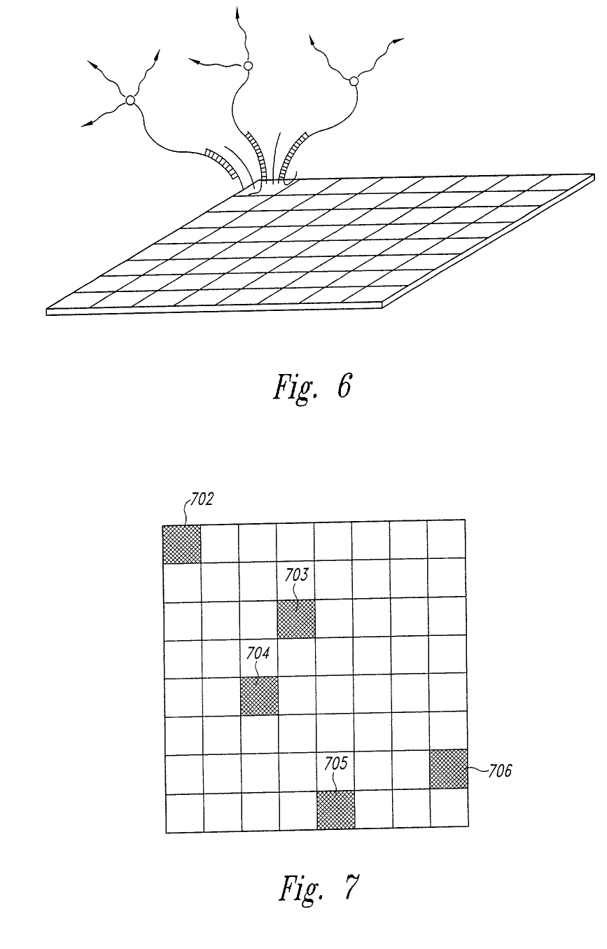

### DETAILED DESCRIPTION OF THE INVENTION

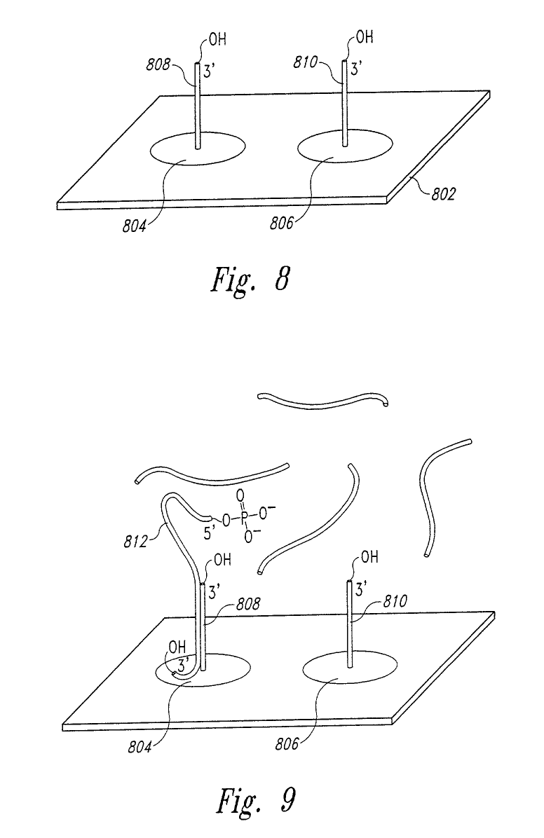

The present invention is related to generation and amplification of signals from features of an array to which target molecules have bound through specific molecular interactions during an experimental, diagnostic, or other analytical procedure. While many commonly employed signal generation techniques require labeling of target molecules prior to hybridization with probe molecules bound to features of an array, the present invention generates and amplifies signals following binding of unlabeled target molecules to probe molecules within features of an array. One embodiment of the present invention is described, in great detail, with reference to FIGS. 8-20. It should be noted that many alternative embodiments are possible, and alternative approaches will be mentioned throughout the description of the preferred embodiment. Finally, a somewhat generalized flow-control diagram of many related embodiments is presented.

FIG. 8 shows a small portion of an array prior to exposure of the array to a sample solution containing target molecules. The portion of the array 802 shown in FIG. 8 contains two features 804 and 806. A single probe DNA oligonucleotide 808 is synthesized on feature 804, and a single probe DNA oligonucleotide 810 is synthesized on feature 806. It is worthwhile to note that probe synthesis takes place 5′ to 3′ by virtue of reverse phosphoramidite chemistry with the latter end pointing away from the plane of the array. Of course, an actual array feature may contain from many thousands to huge numbers of probe molecules, but, for clarity of illustration, only a single probe molecule is shown bound to each feature in FIGS. 8-19. It is assumed that the sequence of probe molecule 808 is different from that of probe molecule 810.

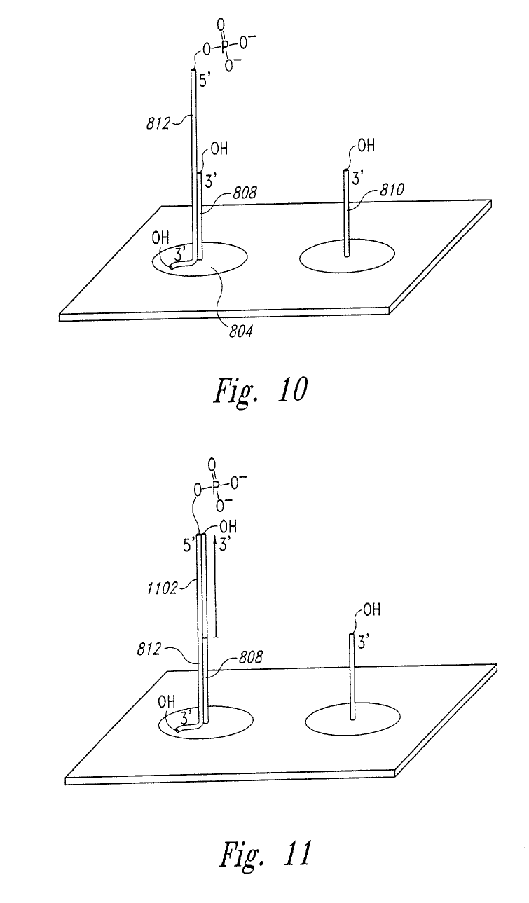

FIG. 9 illustrates exposure of the portion of the array to a sample solution containing amplified, unlabeled target RNA molecules. In FIG. 9, a target molecule 812 containing a region of bases complementary to probe molecule 808 is shown hybridized to probe molecule 808. In the illustrated experiment, there are no target RNA molecules in the sample solution complementary to probe molecule 810 of feature 806.

FIG. 10 illustrates the portion of the array following rinsing of the unbound molecules in solution. In FIG. 10, target molecule 812 remains hybridized to probe molecule 808 of feature 804, while probe molecule 810 remains unhybridized. The state of the array illustrated in FIG. 10 is that arrived at by commonly employed experimental techniques currently used, except for the fact that target molecule 812 is unlabeled and that the probes are synthesized 5′ to 3′. Thus, the portion of the array shown in FIG. 10 contains no signal-emitting chemical entities, even though target molecules have successfully hybridized to probe molecules.

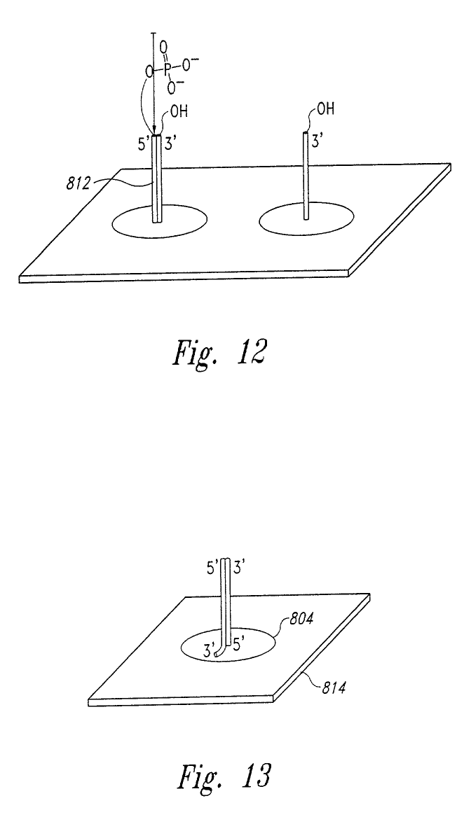

FIG. 11 illustrates a first step in successfully generating a signal from the above experiment. FIG. 12 illustrates a second alternative first step. In FIG. 11, the well-known arrayed primer extension (“APEX”) technique is employed to extend the 3′ end of the probe molecule 808 to equal the length of the single-stranded arm 1102 of the target RNA molecule 812. The APEX technique relies on template-directed polynucleotide extension by DNA polymerase in the presence of the four deoxynucleotide triphosphates. The APEX technique is employed in order to produce a blunt-ended target/probe double-stranded duplex.

In FIG. 12, an alternative approach is taken. Instead of elongating the 3′ terminus of the probe oligonucleotide, the single-stranded 5′ arm of the target molecule 812 is removed under suitable conditions using a 5′-3′ exonuclease, or, alternatively, using single-strand-specific chemical digestion.

FIG. 13 shows the blunt-ended target/probe double-stranded duplex following either probe elongation or target 5′ single-stranded-arm removal. In FIGS. 13-19, only a portion of array 814 including feature 804 is shown, because subsequent steps are directed to modification of double-stranded target/probe hybrids, and do not effect or modify the unhybridized probe molecules of features that fail to bind complementary target molecules. Thus, selection of features to which signal generation and amplification are subsequently applied is effected by preparing blunt-ended target/probe hybrids on those features. As in previous methodologies, hybridization of target molecules to features ultimately produces signals from the features, but while currently available methodologies produce signals directly from labels incorporated in, or bound to, target molecules, the present invention adds signal-emitting chemical entities to target/probe hybrids following hybridization of target molecules to probe molecules.

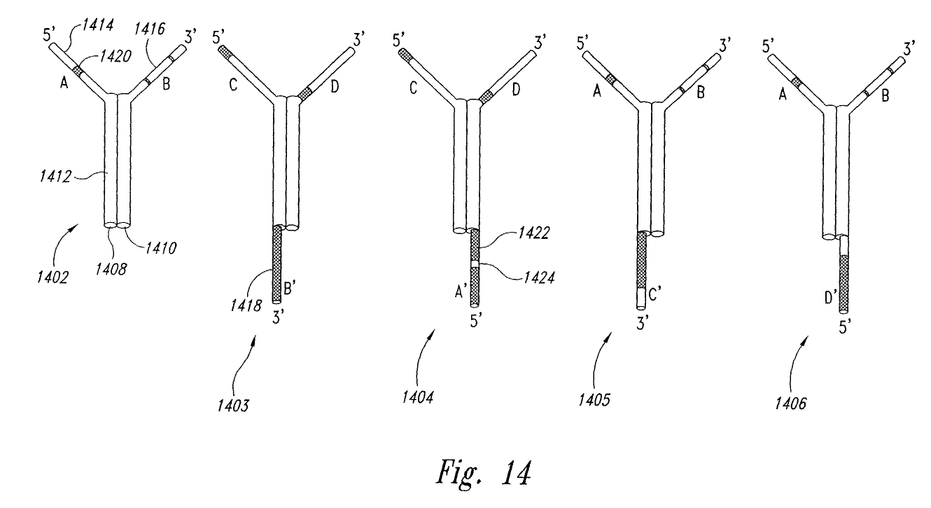

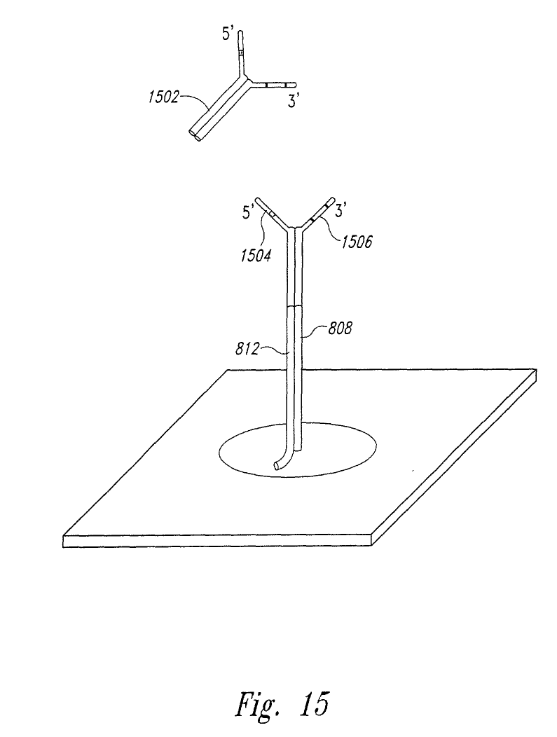

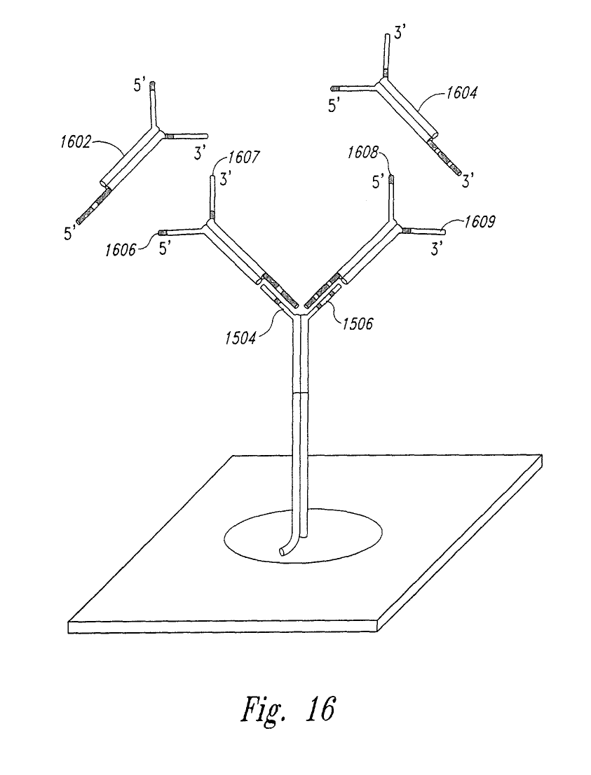

The signal-emitting chemical entities used to generate signals from features containing target/probe hybrids, for one embodiment of the present invention, are shown in FIG. 14. It is important to note that an almost limitless number of signal-emitting linker molecules suitable for use in the signal generation and amplification technique of the present invention can be devised. The detailed chemical identities of any particular labeled oligonucleotide linkers are largely irrelevant. Important, however, is the overall structure of the labeled oligonucleotide linkers and the base complementarity between single-stranded arms of the labeled oligonucleotide linkers. These base-complementarity relationships are described below, with reference to FIG. 14.

In general, each of the linker oligonucleotides shown in FIG. 14 contains one or more chemical labels, such as dye molecules including fluorescein, Cy3, Texas Red, and Cy5 radionuclides, or chemical entities that generate other types of detectable signals, including magnetic signals. The strength of the signal produced through the techniques of the present invention can be controlled, adapted, and fine-tuned by varying the number of signal-emitting chemical entities incorporated in, or bound to, each oligonucleotide linker. Each of the oligonucleotide linkers 1402-1406 comprises a first single-stranded DNA nucleotide polymer having first and second non-complementary regions and a second, anti-parallel DNA polynucleotide polymer having a first region that is base-sequence-complementary to the first region of the first DNA polynucleotide polymer and a second region non-complementary to either the first or second regions of the first DNA polynucleotide polymer or to the first region of the second DNA polynucleotide polymer. For example, oligonucleotide linker 1402 includes a first single-stranded DNA polynucleotide 1408 and a second single-stranded DNA polynucleotide 1410 oriented anti-parallel to the first single-stranded DNA polynucleotide 1408. The base complementary regions of the single-stranded DNA polynucleotides 1408 and 1410 hybridize to form a double-stranded helical region 1412, referred to as the “body” of the oligonucleotide linker. Hybridization and annealing of the complementary regions of oligonucleotide linker 1402 is performed by heating a solution containing 200 μM of each of polynucleotides 1408 and 1410 in the presence of 10 mM NaCl, 5 mM MgCl₂ to 90° C. and slowly cooling it to room temperature over a period of 3 hours. The annealed oligonucleotide linker 1402 is placed on ice and stored frozen. The non-complementary regions of the two DNA polynucleotides do not hybridize, thus forming two single-stranded arms 1414 and 1416. For all but the first primer oligonucleotide linker 1402, the oligonucleotide linkers include a third, single-stranded arm at the opposite end of the linker body from the end containing two single-stranded arms, for example, the third single-stranded arm 1418 of linker oligonucleotide 1403. Annealing and hybridizations of linkers 1403-1406 are carried out as described above using the appropriate combination of single stranded oligonucleotides.

As pointed out above, the actual base sequences of the single-stranded polynucleotides that together comprise an oligonucleotide linker are relevant only in their complementarity to matching regions of other polynucleotides. For example, the base sequence of the first portion of polynucleotide 1408 must be complementary to that of the matching first portion of polynucleotide 1410 in oligonucleotide linker 1402. The actual sequences are unimportant, providing that their base-sequence complementary leads to stable hybridization, as show in FIG. 14.

While the polynucleotides of the double-stranded regions of an oligonucleotide linker must be internally complementary in order to produce the double-stranded body of an oligonucleotide linker, the sequences of single-stranded arms need to bear complementarity relationships with the sequences of single-stranded arms of one or more other oligonucleotide linkers. In FIG. 14, the complementarity relationships are indicated by a graphical code, or markings, on the single-stranded arms of the oligonucleotide linkers. For example, the base sequence labeled A of the single-stranded arm 1404 of oligonucleotide linker 1402 is indicated by a single dark, central stripe 1420. A base sequence complementary to the base sequence A is designated as sequence A′ 422 and is graphically indicated by reversing the colorations of the striped and non-striped portions of the graphical representation of the corresponding single-stranded arm. For example, dark colored stripe 1420 of sequence A is, in sequence A′ 1422, a white-colored stripe 1424. In the following text, the labeled oligonucleotide linkers are referred to by the single-letter designators for their respective single-stranded arm sequences. Thus, primer oligonucleotide linker 1402 is referred to as linker AB, and oligonucleotide linkers 1403-1406 are referred to as CDB′, CDA′, ABC′, and ABD′, respectively. As shown graphically in FIG. 14, sequence A is complementary to sequence A′, sequence B is complementary to B′, sequence C is complementary to C′, and sequence D is complementary to sequence D′. Note that all complementarity relationships are based on standard, anti-parallel, Watson-Crick hybridization.

FIG. 15 illustrates attachment of a primary oligonucleotide linker to the blunt-ended, double-stranded target/probe hybrid bound to a feature of an array. In FIG. 15, the surface of the array is exposed to ligation solution I containing 20 nM oligonucleotide linker 1402, 30 mM Tris-HCl (pH 7.8), 10 mM MgCl2, 10 mM dithiothreitol, 10 mM ATP, and 20 units of T4 DNA ligase. The DNA ligase covalently joins the blunt ends of the double-stranded region of the primer oligonucleotide linker AB (1402 in FIG. 14) to the blunt ends of the target/probe pair 812 and 808. Following the DNA-ligase-mediated reaction, the blunt-ended unlabeled target/probe hybrid pair is converted into a labeled molecule with two single-stranded arms having sequences A 1504 (which is the same as 1404) and B 1506 (which is the same as 1416). The DNA ligase and other solution components are rinsed from the surface of the array to complete the priming step.

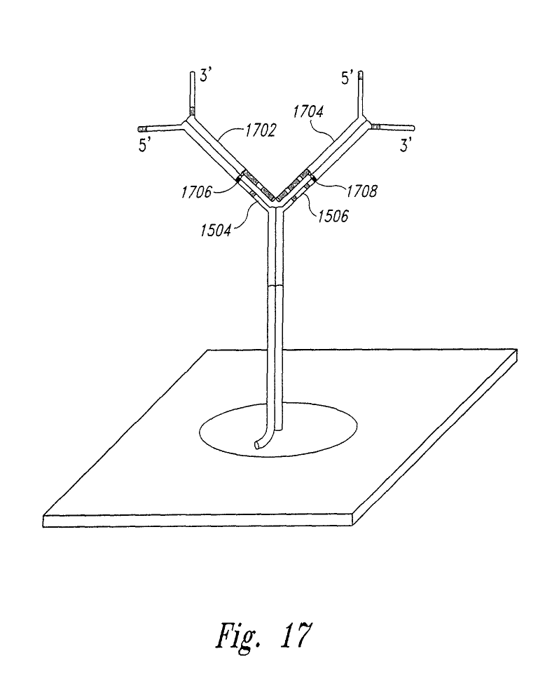

FIG. 16 shows a first amplification step in the described embodiment of the present invention. In FIG. 16, the surface of the array is exposed to a ligation solution II containing 20 nM oligonucleotide linker CDA′ 1602 (1403 in FIG. 14), 20 nM oligonucleotide linker CDB′ 1604 (1404 in FIG. 14), 30 mM Tris-HCl (pH 7.8), 10 mM MgCl₂, 10 mM dithiothreitol, 10 mM ATP, and 20 units of T4 DNA ligase. The third arms of the labeled oligonucleotide linkers hybridize by base complementarity to the single-stranded arms 1504 and 1506 of the primer oligonucleotide linker, as shown in FIG. 16, to produce a topographically Y-shaped branching molecular complex having four unhybridized, non-complementary single-stranded arms 1606-1609. Following hybridization of oligonucleotide linkers CDB′ and CDA′ to the single-stranded arms of the primer oligonucleotide linker, as shown in FIG. 16, the A and B arms of the primer oligonucleotide linker AB are covalently bound to the A′ and B′ arms of the oligonucleotide linkers CDA′ and CDB′, respectively, via the T4 DNA ligase. FIG. 17 shows the resulting topographically Y-shaped molecular entity following ligation. The covalent linkages between the primer linker arms 1504 and 1506 and the hybridized oligonucleotide linkers 1702 and 1704 are indicated in FIG. 17 by dark-colored regions 1706 and 1708. The surface of the array is then rinsed to remove unhybridized oligonucleotide linkers, DNA ligase, cofactors, substrates, and buffering agents.

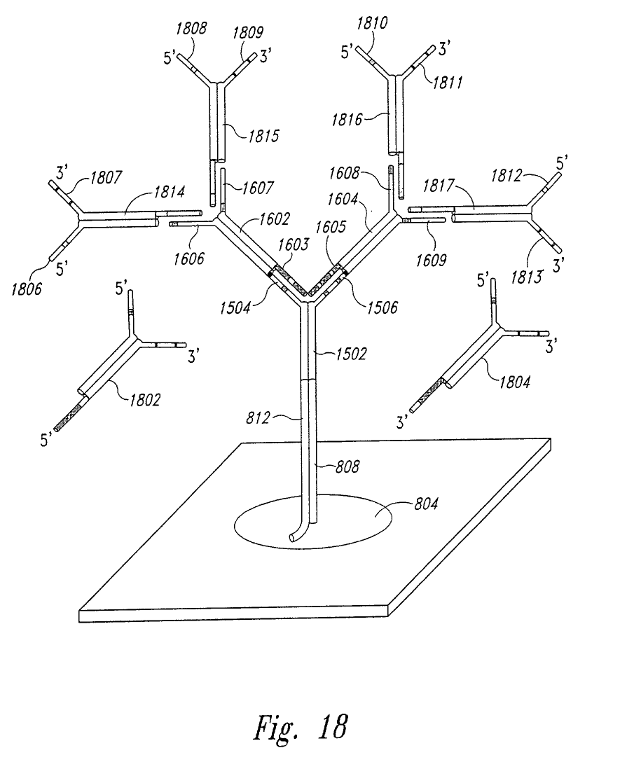

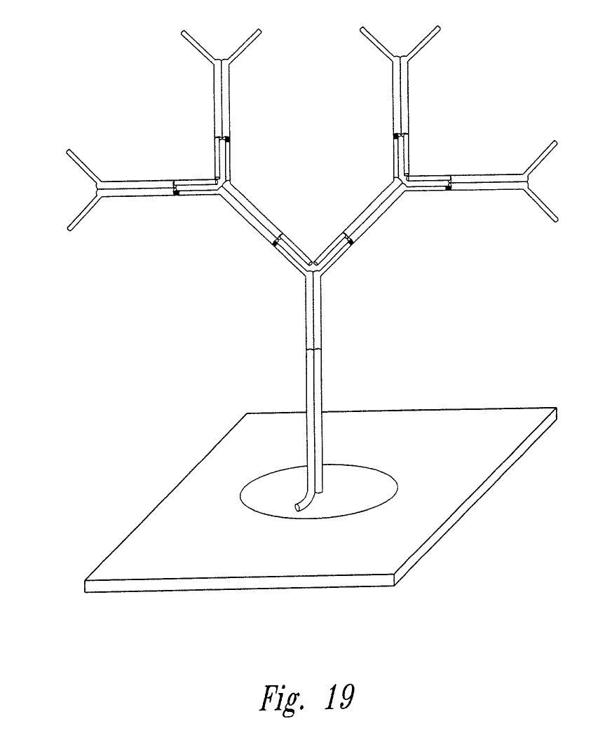

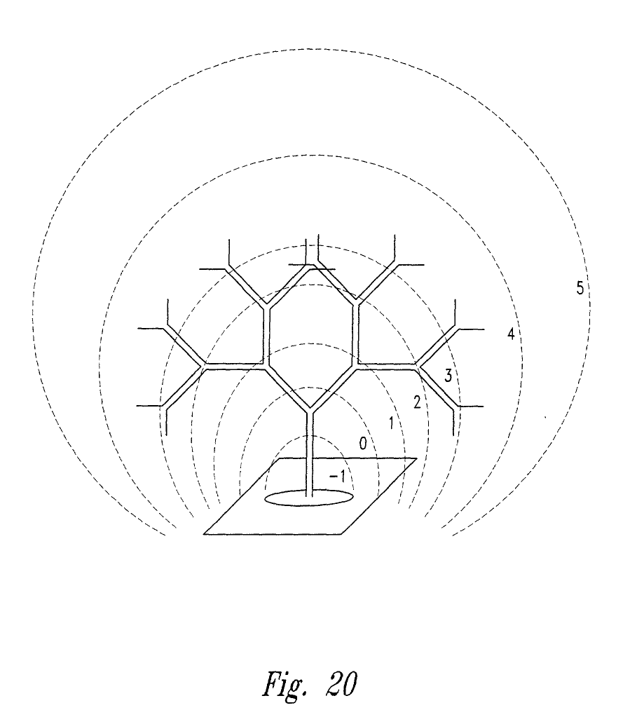

FIG. 18 illustrates addition of yet another layer of oligonucleotide linkers to the branching molecular complex covalently linked to the target/probe hybrid. The surface of the array, in FIG. 18, is exposed to ligation solution III containing 20 nM oligonucleotide linker ABC′ 1804 (1405 in FIG. 14), 20 nM oligonucleotide linker ABD′ 1802 (1406 in FIG. 14), 30 mM Tris-HCl (pH 7.8), 10 mM MgCl₂, 10 mM dithiothreitol, 10 mM ATP, and 20 units of T4 DNA ligase. The D′ and C′ single-stranded arms of these two oligonucleotide linkers 1802 and 1804 hybridize with the D and C arms of the previously added oligonucleotide linkers to form a branching complex with eight unhybridized, single-stranded arms 1806-1815. Note that, in the described embodiment, each new addition of an oligonucleotide linker layer to the complex increases the signal-emitting entities bound to the target/probe hybrid by a factor of two, providing that the number of signal-emitting entities bound to, or incorporated within, the oligonucleotide linkers in each layer is identical. Thus, taking ligation of the primary oligonucleotide linker to the target/probe hybrid as shell zero, the concentration of signal-emitting chemical entities within the branching molecular complex is proportional to 2ⁿ and the number of free, unhybridized single-stranded arms at the surface of the branching molecular structure is 2ⁿ⁺¹, where n is the number of shells. As in the previous addition of oligonucleotide linkers, the hybridized oligonucleotide linkers shown in FIG. 18 are then covalently bound to the arms of the previously added oligonucleotide linkers via the DNA-ligase-mediated reaction. FIG. 19 shows the branching molecular complex shown in FIG. 18 following ligation.

FIG. 20 abstractly represents additional layers, or shells, of oligonucleotide linkers added to the covalently bound branching complex to further increase the size of the complex. This dendrimer-like complex can be grown to arbitrary sizes in order to produce a sufficient number of signal-emitting chemical entities bound to the features of the array. Note that the local concentration of signal-emitting entities can be controlled both by the number of shells, or layers, of labeled oligonucleotide linkers as well as by the number of signal-producing chemical entities bound to, or incorporated within, the labeled oligonucleotide linkers.

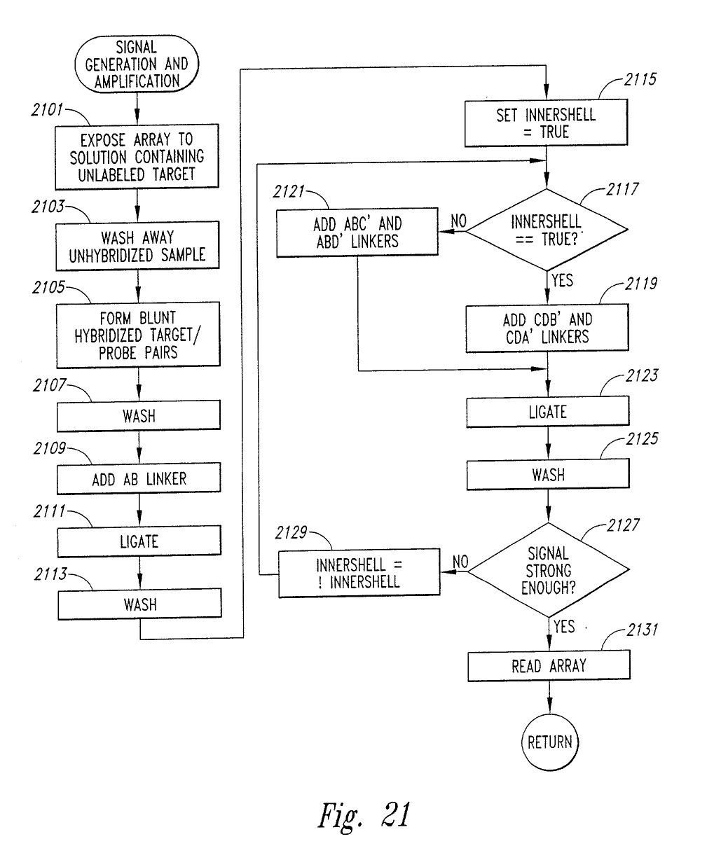

FIG. 21 is a flow-control diagram that describes multiple alternative embodiments of the present invention in a more algorithmic fashion. In step 2101, a prepared array is exposed to a solution containing unlabeled target molecules, discussed with respect to the above-described embodiment with reference to FIG. 9. In step 2103, the sample solution is rinsed from the surface of the array. In step 2105, the target/probe hybrids formed by anti-parallel, complementary base pairing between target molecules and probe molecules bound to the array are processed to form blunt ends, as discussed above with reference to FIGS. 11-13. In step 2107, the reactants employed to form blunt ends are rinsed from the surface of the array. In step 2109, the AB primer linker (1402 in FIG. 14) is added to the surface of the array and, in step 2111, the AB primer linker is covalently bound to the blunt ends of the target/probe hybrids via a DNA-ligase-mediated reaction. In step 2113, unreacted primer linker molecules, DNA ligase, and other cofactors, substrates, and buffer agents are washed form the surface of the array.

Steps 2115-2129 form a control loop, or iterative inner process, in which layers of oligonucleotide linkers are added to the dendrimer-like molecular complex bound to target/probe hybrids. In step 2115, a loop-control variable “innershell” is set to Boolean value TRUE. In step 2117, the current value of the loop-control variable “innershell” is determined. If the current value is TRUE, then a solution containing oligonucleotide linkers “CDB′” and “CDA”′ (1403 and 1404 in FIG. 14) is added to the surface of the array. Otherwise, a solution containing oligonucleotide linkers “ABC”′ and “ABD”′ (1405 and 1406 in FIG. 14) is added to the surface of the array in step 2121. Then, the added oligonucleotide linkers are covalently bound to the ends of the previously added oligonucleotide linkers in step 2123. In step 2125, the ligation mediator, cofactors, substrates, and buffering agents, as well as unhybridized oligonucleotide linkers, are washed from the surface of the array. In step 2127, the signal strength or calculated signal strength resulting from the current dendrimer-like branching complex is determined. If the signal strength is insufficient for analytical purposes, then, in step 2129, the loop-control variable “innershell” is assigned to the opposite value from its current value via a Boolean NOT operation, and control flows back to step 2117. Otherwise, sufficient signal generation and amplification has been carried out by techniques of the present invention, and the array may be analyzed in step 2131.

Although the present invention has been described in terms of a particular embodiment, it is not intended that the invention be limited to this embodiment. Modifications within the spirit of the invention will be apparent to those skilled in the art. For example, while the described embodiment concerns probe DNA polynucleotides complementary to target RNA polynucleotides, the present invention is applicable to other types of probe/target biopolymers, synthetic polymers, and other types of compounds that bind to specific recognition sites. For example, synthetic nucleotide polymers having thioester rather than phosphodiester backbones, modified sugars, or non-standard bases may be synthesized and may hybridize to other polynucleotides through complementary base pairing. Additional specific recognition may include protein binding to polynucleotides, protein/protein binding, antigen-to-antibody binding, and other such specific molecular recognition and association. Furthermore, even when applied to polynucleotides, there are an almost limitless number of alternative embodiments employing different primer oligonucleotide linkers and oligonucleotide linkers, including varying the base sequences and polymer lengths of the double-stranded regions, or bodies, of the oligonucleotide linkers as well as the lengths and nucleotide sequences of the arms. Furthermore, additional sets of oligonucleotide linkers can be incorporated into the techniques of the present invention so that, rather than employing two alternating sets of oligonucleotides in the iterative inner-process loop of steps 2117-2129 in FIG. 21, three or more sets of oligonucleotide linkers may be added, in sequence, during each iteration. More complex, branching oligonucleotide linkers may be devised. As discussed above, a number of different signal-emitting chemical entities can be use to label the labeled oligonucleotide linkers, including chemical dyes, chemical entities with specific magnetic properties that can be detected instrumentally, chemoluminescent and fluorescent entities, and other signal-emitting or otherwise detectable entities. The amplification process may be carried out for a specific number of iterations, or shell additions, or may employ signal detection means to detect when a sufficiently strong signal is generated from features of an array.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for binding signal-emitting entities to target/probe molecule pairs within features of an array, the method comprising:
binding primer linker molecules to target/probe molecule pairs within features of the array to form nascent complexes bound to target/probe molecule pairs; and
repeatedly binding additional linker molecules that include one or more signal-emitting entities to the nascent complexes until molecular complexes containing a sufficient number of signal-emitting entities are obtained.

2. The method of claim 1 wherein the complexes contain a sufficient number of signal-emitting entities when a signal can be detected from a feature containing target/probe molecular pairs bound to the complexes.

3. The method of claim 1 wherein repeatedly binding additional linker molecules that include one or more signal-emitting entities to the nascent complexes further includes:
binding a first set of linker molecules including one or more signal-emitting entities to linker molecules previously bound to the complexes; and
binding a second set of linker molecules including one or more signal-emitting entities to linker molecules of the first set of linker molecules previously bound to the complexes.

4. Results, stored in a computer-readable medium, produced by detecting signals from an array to which signal-emitting entities are bound to target/probe pairs by the method of claim 1.

5. Results, transferred to an intercommunicating entity via electronic signals, produced by detecting signals from an array to which signal-emitting entities are bound to target/probe pairs by the method of claim 1.

6. Results, printed in a human-readable format, produced by detecting signals from an array to which signal-emitting entities are bound to target/probe pairs by the method of claim 1.

7. A system for generating and amplifying signals from target/probe molecule pairs bound to an array, the system comprising:
an array having features containing target/probe molecule pairs;
a primer linker solution used to bind a primer linker to target/probe molecule pairs to generate a nascent molecular complex;
a solution containing a first set of linker molecules that include signal-emitting entities used to associate linker molecules of the first set of linker molecules to linker molecules previously incorporated in the complex;
a solution containing a second set of linker molecules that include signal-emitting entities used to associate linker molecules of the second set of linker molecules to linker molecules previously incorporated in the complex; and
a covalent-binding mediator that mediates covalent binding of associated linker molecules.

8. A method for binding signal-emitting entities to a complementary polynucleotide-target/polynucleotide-probe pair bound to an array, the method comprising:
generating a blunt end at an unbound terminus of the polynucleotide-target/polynucleotide-probe pair;
binding a primer oligonucleotide linker to the blunt end of the unbound terminus of the polynucleotide-target/polynucleotide-probe pair to form a complex; and
repeatedly binding oligonucleotide linkers including signal-emitting entities to oligonucleotide linkers previously bound to the complex.

9. The method of claim 8 wherein the complementary polynucleotide-target/polynucleotide-probe pair comprises an RNA target molecule hybridized to a probe DNA oligonucleotide.

10. The method of claim 9 wherein generating a blunt end at an unbound terminus of the polynucleotide-target/polynucleotide-probe pair further includes applying an arrayed primer extension technique to extend the probe molecule.

11. The method of claim 9 wherein generating a blunt end at an unbound terminus of the polynucleotide-target/polynucleotide-probe molecule pair further includes applying an exonuclease to digest unhybridized single-stranded regions of the target molecule.

12. The method of claim 9 wherein oligonucleotide linkers each comprises:
a first single-stranded DNA nucleotide polymer having first and second noncomplementary regions; and
a second, anti-parallel DNA polynucleotide polymer having a first region that is base-sequence-complementary to the first region of the first DNA polynucleotide polymer and a second region non-complementary to either the first or second regions of the first DNA polynucleotide polymer and to the first region of the second DNA polynucleotide polymer, the base complementary regions of the first and second single-stranded DNA polynucleotides hybridizing to form a double-stranded body and the non-complementary regions of the first and second single-stranded DNA polynucleotides forming two single-stranded arms.

13. The method of claim 12 wherein non-primer oligonucleotide linkers each further comprises a third, single-stranded arm at an opposite end of the linker body from the two single-stranded arms.

14. The method of claim 13
wherein the primer oligonucleotide linker includes a first arm having a first base sequence and a second arm having a second base sequence; and
wherein non-primer oligonucleotide linkers include
a first set of non-primer oligonucleotide linkers that includes
an oligonucleotide linker having a first arm with a third base sequence, a second arm with a fourth base sequence, and a third arm with a base sequence complementary to the first base sequence, and
an oligonucleotide linker having a first arm with a third base sequence, a second arm with a fourth base sequence, and a third arm with a base sequence complementary to the second base sequence; and
a second set of non-primer oligonucleotide linkers that includes
an oligonucleotide linker having a first arm with the first base sequence, a second arm with the second base sequence, and a third arm with a base sequence complementary to the third base sequence, and
an oligonucleotide linker having a first arm with the first base sequence, a second arm with the second base sequence, and a third arm with a base sequence complementary to the fourth base sequence.

15. The method of claim 14 wherein repeatedly binding oligonucleotide linkers including signal-emitting entities to oligonucleotide linkers previously bound to the complex further includes:
binding non-primer oligonucleotide linkers of the first set of oligonucleotide linkers to the molecular complex via hybridization of the third arms of the non-primer oligonucleotide linkers of the first set of oligonucleotide linkers to complementary arms extending from the complex; and
binding non-primer oligonucleotide linkers of the second set of oligonucleotide linkers to the molecular complex via hybridization of the third arms of the non-primer oligonucleotide linkers of the second set of oligonucleotide linkers to complementary arms extending from the complex.

16. A method for covalently binding layers of partially double-stranded oligonucleotide linkers onto a polynucleotide-target/polynucleotide-probe pair bound to an array to form a complex that can be detected by analysis, the method comprising:
covalently binding an initial partially double-stranded oligonucleotide linker to the polynucleotide-target/polynucleotide-probe pair, the initial partially double-stranded oligonucleotide linker having at least two single-stranded oligonucleotide arms and forming a complex with as least two single-stranded arms; and
repeatedly
covalently binding one or more next partially double-stranded oligonucleotide linkers to the complex following association of the single-stranded oligonucleotide arms of the complex to complementary single-stranded arms of the next partially double-stranded oligonucleotide linkers, each one or more next double-stranded oligonucleotide linker having a single-stranded oligonucleotide arm complementary to the to one or more of the single-stranded oligonucleotide arms of the complex and at least one single-stranded oligonucleotide arm not complementary to the single-stranded oligonucleotide arms of the complex and not complementary to the single-stranded oligonucleotide arms of the one or more next double-stranded oligonucleotide linkers.

17. The method of claim 16 wherein the initial partially double-stranded oligonucleotide linker has a blunt, double-stranded end and two single-stranded oligonucleotide arms at the end opposite from the blunt, double-stranded end.

18. The method of claim 16 wherein covalently binding an initial partially double-stranded oligonucleotide linker to the polynucleotide-target/polynucleotide-probe pair an further comprises:
forming a blunt, free end on the polynucleotide-target/polynucleotide-probe pair; and
ligating the blunt end of the initial partially double-stranded oligonucleotide linker to the blunt, free end of the polynucleotide-target/polynucleotide-probe pair.

19. The method of claim 16 wherein each one or more next partially double-stranded oligonucleotide linker has a first end having one single-stranded oligonucleotide arm complementary to the to one or more of the single-stranded oligonucleotide arms of the complex, a double-stranded body, and a second end having two single-stranded oligonucleotide arms not complementary to the single-stranded oligonucleotide arms of the complex and not complementary to the single-stranded oligonucleotide arms of the one or more next double-stranded oligonucleotide linkers.

20. The method of claim 19 wherein repeatedly covalently binding one or more next partially double-stranded oligonucleotide linkers to the complex following association of the single-stranded oligonucleotide arms of the complex to complementary single-stranded arms of the next partially double-stranded oligonucleotide linkers further includes:
repeatedly
covalently binding a first set of partially double-stranded oligonucleotide linkers to the complex; and
covalently binding a second set of partially double-stranded oligonucleotide linkers to the complex.

21. The method of claim 19 wherein, after covalent binding of one or more next partially double-stranded oligonucleotide linkers to a complex with n single-stranded oligonucleotide arms, the resulting complex has approximately 2n single-stranded oligonucleotide arms.

22. Results, stored in a computer-readable medium, produced by analyzing an array containing complexes generated by the method of claim 16.

23. Results, transferred to an intercommunicating entity via electronic signals, produced by analyzing an array containing complexes generated by the method of claim 16.

24. Results, printed in a human-readable format, produced by analyzing an array containing complexes generated by the method of claim 16.
